(19) United States
(12) Patent Application Publication
(10) Pub. No.: US 2009/0118804 A1
(43) Pub. Date: May 7, 2009

(54) METHOD OF MOUNTING MINIMALLY INVASIVE PLUG ELECTRODES WITHIN CRANIUM OF PATIENT

(75) Inventors: Michael Adam Moffitt, Valencia, CA (US); Jeffery Van Funderburk, Stevenson Ranch, CA (US)

Correspondence Address: Vista IP Law Group LLP, 2040 MAIN STREET, 9TH FLOOR, IRVINE, CA, 92614, US

(73) Assignee: ADVANCED BIONICS CORPORATION, Valencia, CA (US)

(21) Appl. No.: 11/935,368

(22) Filed: Nov. 5, 2007

(51) Int. Cl. A61N 1/05

(52) U.S. Cl. 607/116

(57) ABSTRACT

A method of performing a medical procedure on a patient comprises forming a burr hole through the cranium of the patient, mounting a permanently integrated plug electrode within the burr hole, and electrically coupling the plug electrode to an electronics device. Another method of performing a medical procedure on a patient comprises forming a burr hole through the cranium of the patient, mounting an electrode within the burr hole, such that the electrode does not extend within the brain of the patient, and electrically coupling the electrode to an electronics device. A hybrid plug/electrode comprises a plug body configured for being anchored within a burr hole formed within a cranium of a patient, at least one electrode disposed on a distal-facing surface of the plug body, and at least one electrode lead affixed within the plug body in electrical communication with the at least one electrode.

### FIELD OF THE INVENTION

The present inventions relate to burr hole plugs used to seal and secure electrical stimulation leads and electrodes within a cranial burr hole.

### BACKGROUND OF THE INVENTION

Implantable neurostimulation systems have proven therapeutic in a wide variety of diseases and disorders. For example, it is known to use such systems to treat neurological disorders, such as neurodegenerative diseases (e.g., Alzheimer's Disease, Parkinson's Disease, tremor, and epilepsy), brain ischemia, such as stroke, and limbic disorders, as well as non-neurological disorders, such as migraine headaches, obesity, and pain syndromes (such as trigeminal neuralgia) by electrically stimulating selected portions of the brain. While deep brain stimulation (DBS) procedures have been the focus of attention in treating many of these neurological disorders, there have been some considerable developments in cortical brain stimulation procedures, wherein the cortical brain tissue is stimulated to rehabilitate stroke victims, provide pain relief, as well as to provide benefits in the treatment of the other aforementioned disorders.

A typical implantable neurostimulation system used to electrically stimulate brain tissue includes electrodes, which are implanted at the desired stimulation site in the brain of the patient (in the case of cortical brain stimulation, along the cortex of the brain), and a neurostimulator implanted remotely from the stimulation site (e.g., in the chest region of the patient), but coupled either directly to the electrodes via one or more leads. The neurostimulation system may further comprise a handheld remote control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon.

In cortical stimulation procedures, it is typically necessary to place a variety of stimulation electrodes, as well as recording electrodes, along the surface of the cortex. Thus, to provide broad access to the cortex, a craniotomy, which is a relatively invasive procedure that involves removing a large portion of the cranium (referred to as a “turning a bone flap”) and then putting the bone flap back into place after the electrodes have been affixed along the cortex, must be performed on the patient. Alternatively, multiple burr holes can be meticulously cut through the cranium, so that the stimulation/recording electrodes can be placed through the burr holes into contact with the various target sites of the cortex. Titanium or stainless steel bands or a cranial burr hole plug can then be installed over or within each burr hole used during the implantation procedure to hold the electrode in place, as well as to seal the burr hole. A typical burr hole plug includes a multitude of components, including a ring-shaped base that is anchored to the cranium typically using screws, retainer that is integrated with the plug base to secure the electrode in place, and a cap that fits over the plug base to seal the burr hole and/or further secure the electrode in place.

While providing access to the various target sites of the brain cortex using multiple burr holes is less invasive than performing a craniotomy, the size of the burr holes are still relatively large (typically, 14-15 mm in diameter). In addition to meticulously drilling each burr hole in the cranium, the different components of each burr hole plug must be assembled within the respective burr hole, while maintaining the stimulation lead in place, thereby further increasing the procedure time.

There, thus, remains a need for a less invasive and efficient means for providing access to, and implanting electrodes adjacent the brain of a patient.

### SUMMARY OF THE INVENTION

In accordance with a first aspect of the present inventions, a method of performing a medical procedure on a patient is provided. The method comprises forming a burr hole through the cranium of the patient. To minimize the invasiveness of the medical procedure, the diameter of the burr hole may be less than 10 mm, and even less than 5 mm. The method further comprises mounting an integrated plug electrode within the burr hole (e.g., by screwing the plug electrode into the burr hole).

In one method, the plug electrode has at least one electrode formed on an external surface of the plug electrode. In another method, the plug electrode has at least one electrode that is disposed within the burr hole when the plug electrode is mounted within the burr hole. In still another method, a proximal portion of the plug electrode is electrically insulated from the patient, and a distal portion of the plug electrode is exposed to form at least one electrode. In yet another method, the burr hole is formed within the cranium of the patient and the plug electrode is mounted within the burr hole by screwing the plug electrode directly into the cranium of the patent.

The method further comprises electrically coupling the plug electrode to an electronics device (e.g., a neurostimulator and/or electrical signal recorder). In one method, electrically coupling the plug electrode to the electronics device comprises connecting an electrical lead between the plug electrode and the electronics device. In an optional method, the electronics device is a neurostimulator, and the method further forming another burr hole through the cranium of the patient, mounting another electrode within the other burr hole, such that the other electrode does not extend within the brain the patient, and electrically coupling the other electrode to an electrical signal recorder.

In accordance with a second aspect of the present inventions, another method of performing a medical procedure on a patient is provided. The method comprises forming a burr hole through the cranium of the patient. To minimize the invasiveness of the medical procedure, the diameter of the burr hole may be less than 10 mm, and even less than 5 mm. The method further comprises mounting an electrode within the burr hole, such that the electrode does not extend into the brain of the patient. In one method, a plug is mounted within the burr hole to secure the electrode within the burr hole. In one example, the plug and electrode form an integrated plug electrode that is mounted within the burr hole to secure the electrode within the burr hole. In another example, the electrode is carried by a lead, and the lead is affixed within the plug to secure the electrode within the burr hole.

The method further comprises electrically coupling the electrode to an electronics device. In an optional method, the electronics device is a neurostimulator, and the method further forming another burr hole through the cranium of the patient, mounting another electrode within the other burr hole, such that the other electrode does not extend within a cranial cavity of the patient, and electrically coupling the other electrode to an electrical signal recorder.

In accordance with a third aspect of the present invention, a hybrid plug/electrode comprises a plug body configured for being anchored within a burr hole formed within a cranium of a patient. In one embodiment, the plug body comprises a cylindrical outer wall configured for engaging an inner surface of the burr hole. In another embodiment, the plug body comprises at least one fastening mechanism (e.g., a thread) disposed on the cylindrical outer wall for anchoring the plug body to the inner surface of the burr hole. The hybrid plug/electrode further comprises at least one electrode disposed on a distal-facing surface of the plug electrode, and at least one electrode lead affixed within the plug body in electrical communication with the electrode(s). In the case where a plurality of electrodes is provided, a plurality of electrical leads can be respectively coupled to the electrodes. The hybrid plug/electrode further comprises a connector affixed to the plug body in electrical communication with the electrode lead(s). The connector is configured for being externally accessible when the plug body is anchored within the burr hole. The connector may be configured for receiving a lead extension.

The hybrid plug/electrode can be used in various systems and methods. For example, a medical system may have the hybrid plug/electrode and an electronics device coupled to the one or more electrode leads. A method of performing a medical procedure on a patient may comprise forming the burr hole through the cranium of the patient, anchoring the hybrid plug/electrode within the burr hole, and electrically coupling the hybrid plug/electrode to an electronics device. Another method of performing a medical procedure on a patient may comprise conveying electrical energy from the electrode(s) of the hybrid plug/electrode to stimulate cortical brain tissue of the patient. Still another method of performing a medical procedure on a patient may comprise recording electrical signals from the cortical brain tissue using the electrode of the hybrid plug/electrode.

Other and further aspects and features of the invention will be evident from reading the following detailed description of the embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a cortical brain stimulation system implanted within a patient;

FIG. 2 is a plan view of exemplary stimulation and recording sites of a patient where electrodes of the cortical brain stimulation system of FIG. 1 may be implanted

FIG. 3 is a cross-sectional view of one embodiment of a hybrid plug/electrode array that can be used in the cortical brain stimulation system of FIG. 1;

FIG. 4 is a cross-sectional view of one embodiment of a minimally invasive plug electrode that can be used in the cortical brain stimulation system of FIG. 1;

FIG. 5 is a cross-sectional view of another embodiment of a minimally invasive plug electrode that can be used in the cortical brain stimulation system of FIG. 1;

FIG. 6 is a cross-sectional view of still another embodiment of a minimally invasive plug electrode that can be used in the cortical brain stimulation system of FIG. 1;

FIG. 7 is a cross-sectional view of yet another embodiment of a minimally invasive plug electrode that can be used in the cortical brain stimulation system of FIG. 1;

FIG. 8 is a cross-sectional view of yet another embodiment of a minimally invasive plug electrode that can be used in the cortical brain stimulation system of FIG. 1; and

FIG. 9 is a top view of the minimally invasive plug electrode of FIG. 8.

### DETAILED DESCRIPTION OF THE EMBODIMENTS

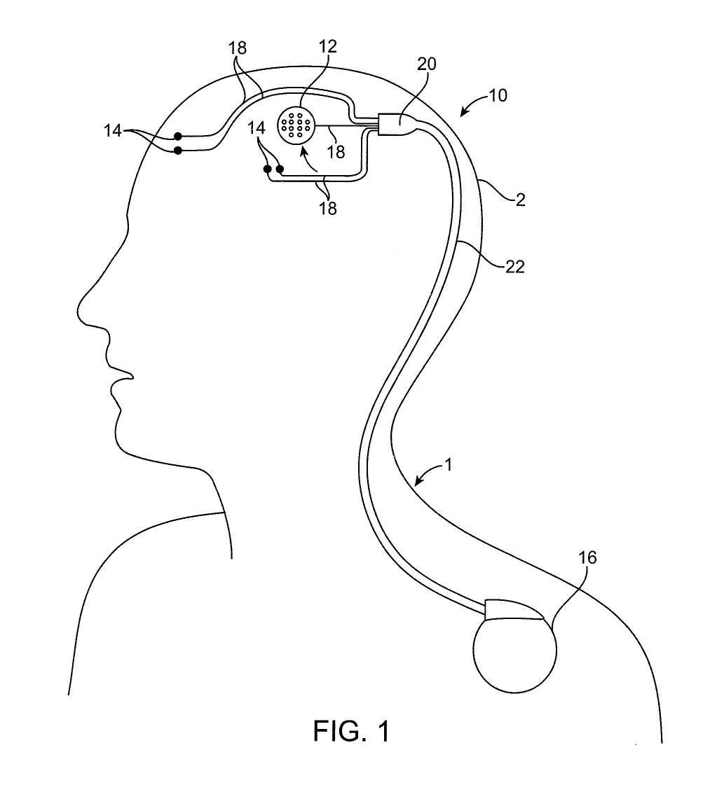

Turning first to FIG. 1, an exemplary cortical brain stimulation system 10 constructed and arranged in accordance with one embodiment of the present inventions is shown implanted within a patient for the treatment of a debilitating disease such as, Parkinson's disease, dystonia, essential tremor, seizure disorders, obesity, depression, etc. The system 10 comprises a hybrid plug/electrode array 12 and a plurality of minimally invasive plug electrodes 14 implanted within the head 2 of a patient 1 for both stimulating and recording electrical signals from the cortical brain tissue (not shown in FIG. 1). Both of the hybrid plug/electrode array 12 and plug electrodes 14 can be considered plug electrodes, with the main difference being that the hybrid plug/electrode array 12 comprises a plurality of electrodes, whereas the plug electrodes 14 comprise a single electrode. As will be described in further detail below, the hybrid plug/electrode array 12 and plug electrodes 14 are mounted within the cranium of the patient in order to stimulate or record signals of the cortical brain tissue.

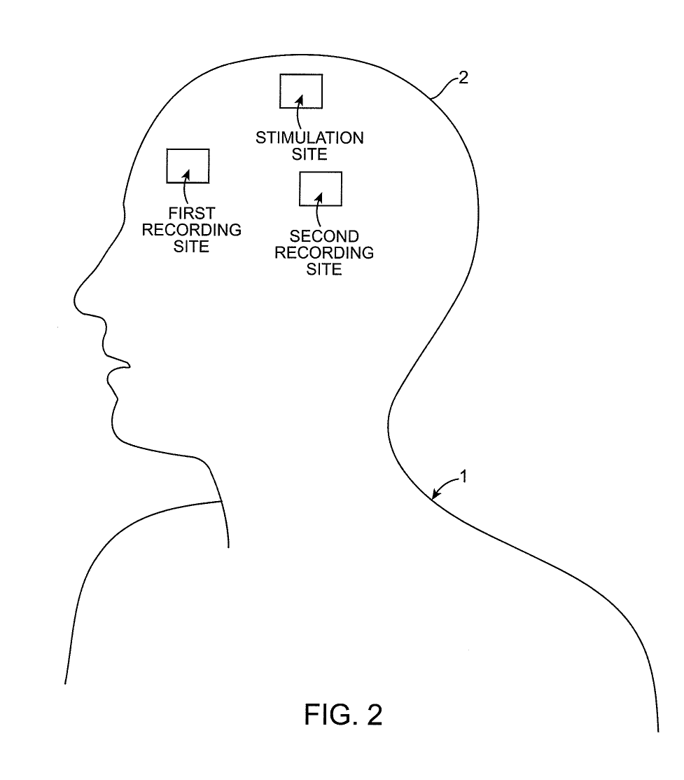

As illustrated in FIG. 2, the hybrid plug/electrode array 12, in one embodiment, is used to stimulate a portion of the cortical brain tissue at a stimulation site, and the plug electrodes 14 are used to record electrical signals at first and second recording sites of the cortical brain tissue. Thus, electrical stimulation energy can be conveyed from the hybrid plug/electrode array 12 into the cortical brain tissue to treat the disease, and electrical signals can be sensed at the plug electrodes 14 to monitor the disease.

Alternatively, the hybrid plug/electrode array 12 can be used to record electrical signals from one of the two recording sites of the cortical brain tissue, and the plug electrodes 14 may be used to convey stimulation energy to the stimulation site of the cortical brain tissue. In another alternative embodiment, some of the plug electrodes 14 may be used to convey stimulation energy to the stimulation site of the cortical brain tissue, and others of the plug electrodes 14 may be used to record electrical signals from one or both of the recording sites of the cortical brain tissue. In an optional embodiment, the electrical signals sensed at the first and second recording sites can be used to modify or adjust stimulation parameters in accordance with which the stimulation energy is delivered from the plug/electrode array 12 to the stimulation site. Further details discussing this closed-loop manner of delivery stimulation energy to a patient are described in U.S. patent application Ser. No. ______ (Attorney Docket No. 06-00363-01), which is expressly incorporated herein by reference.

The cortical brain stimulation system 10 further comprises an implantable electronics device 16. In the illustrated embodiment, the electronics device 16 takes the form of a electronics device 16, such as an implantable pulse generator (IPG), radio frequency (RF) receiver-stimulator, or any other device coupled to and capable of delivering electrical stimulation energy to the hybrid plug/electrode array 12 in a controlled and therapeutic manner. In the illustrated embodiment, the electronics device 16 may also include recording circuitry capable of processing electrical signals sensed at the plug electrodes 14. In this manner, the electronics device 16 can be considered both a neurostimulator and a recorder. Alternatively, separate neurostimulator and recording devices can be utilized. In any event, the cortical brain stimulation system 10 further comprises a plurality of individual electrical leads 18 respectively coupled to the hybrid plug/electrode array 12 and plug electrodes 14, a connector hub 20 that receives the ends of the electrical leads 18, and a lead extension 22 coupled between the connector hub 20 and the electronics device 16. As will be described, the electrical leads 18, in certain cases, may carry electrodes that form portions of the plug electrodes 14.

The connector hub 20 may be implanted underneath the scalp of the patient, and the individual electrical leads 18 may be subcutaneously routed from the hybrid plug/electrode array 12 and plug electrodes 14 underneath the scalp, along external surface of the cranium of the patient, to the connector hub 20. In this manner, the individual electrical leads 18 can be wired through the single lead extension 22. The lead extension 22 may be subcutaneously advanced underneath the scalp of the patient 1 to the electronics device implantation site, thereby facilitating the location of the electronics device 16 away from the cranium of the patient. The electronics device 16 may be generally implanted in a surgically-made pocket in the torso of the patient (e.g., the chest or shoulder region). The electronics device 16 may, of course, also be implanted in other locations of the patient's body. In alternative embodiments, the electronics device 16 may be directly implanted on or within the cranium of the patient 1, as described in U.S. Pat. No. 6,920,359, which is expressly incorporated herein by reference. In this case, the lead extension 22 may not be needed. The system 10 may include external components, such as a patient handheld programmer, a clinician programming station, and an external charger (all not shown), the details of which will not be described herein for purposes of brevity.

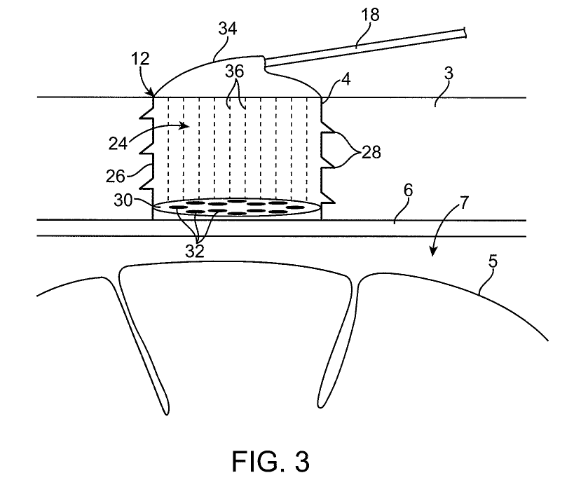

Turning now to FIG. 3, the hybrid plug/electrode array 12 will now be described. As there shown, the hybrid plug/electrode array 12 is mounted within a burr hole 4 conventionally formed through the cranium 3 of the patient 1. The hybrid plug/electrode array 12 comprises a plug body 24 that is sized to firmly fit within the burr hole 4, thereby firmly anchoring the hybrid plug/electrode array 12 to the cranium 3 and preventing leakage of cerebral spinal fluid between the outer surface of the plug body 24 and the burr hole 4. To this end, the plug body 24 has a cylindrical outer wall 26 having an outer diameter substantially the same as the diameter of the burr hole 4. The burr hole 4 may have a conventional size; for example, over 10 mm in diameter, and typically between 14-15 mm in diameter. The plug body 24 comprises a fastener, and in particular, a thread 28 disposed on the outer surface of the cylindrical wall 26, for engaging the inner surface of the burr hole 4, and thereby, anchoring the hybrid plug/electrode array 12 within the burr hole 4. In this manner, the hybrid plug/electrode array 12 may be conveniently screwed into the burr hole 4. Alternatively, other types of fastening means, such as sutures or bone screws, can be used to anchor the hybrid plug/electrode array 12 to the cranium 3.

The plug body 24 further comprises a distal surface 30 that faces the cortical brain tissue 5 of the patient 1 when the hybrid plug/electrode array 12 is anchored within the burr hole 4. In the illustrated embodiment, the distal surface 30 of the plug body 24 is flat, although in alternative embodiments, may be concave or convex. The height of the plug body 24 has a relatively small profile, such that the distal surface 30 does not protrude into the cranial cavity of the patient (i.e., does not extend past the inner surface of the cranium 3) when the hybrid plug/electrode array 12 is anchored within the burr hole 4. For example, as shown in FIG. 3, the distal surface 30 is recessed relative to the inner surface of the cranium 3. In some cases, it may be desirable for the hybrid plug/electrode array 12 to extend into the cranial cavity and sit or push gently on the dura mater, thus placing the active portion of the hybrid plug/electrode array 12 closer to the target neural tissue.

The plug body 24 may be composed of a suitable hard biocompatible material, such as titanium, stainless steel (e.g., MP35N), alloys, or hard polymers (e.g., a high durometer silicone, polyurethane, or polyethertheterketone (PEEK)). If the plug body 24 is composed of an electrically conductive material, the hybrid plug/electrode array 12 may comprise an electrically insulative coating (not shown) disposed on the outer surface of the plug body 24 to ensure that the cranium 3 is electrically insulated from the hybrid plug/electrode array 12 and to minimize noise from electromyograms (EMGs) during recording.

The hybrid plug/electrode array 12 further comprises a plurality of electrodes 32 suitably mounted to the distal surface 30 of the plug body 24, such that the electrodes 32 face the cortical brain tissue 5. As shown, because the distal surface 30 of the plug body 24 is recessed within the burr hole 4, the electrodes 32 are likewise recessed within the burr hole 4. Although the electrodes 32 are not in direct contact with the cortical brain tissue 5, they are still electrically coupled to the cortical brain tissue 5 via the dura mater 6 and cerebrospinal fluid 7. Thus, the electrodes 32 may potentially convey electrical stimulation energy (originating from the electronics device 16) to the cortical brain tissue 5 or receive electrical signals from the cortical brain tissue 5 for subsequent processing in the electronics device 16. The electrodes 32 may be disposed on the distal surface 30 of the plug body 24 in any conventional manner (e.g., electroplating, sputtering, or bonding), and may be composed of any suitable biocompatible, electrically conductive material, such as stainless steel or a platinum alloy.

The hybrid plug/electrode array 12 further comprises an electrical connector 34 and a plurality of electrode leads 36 (shown in phantom) extending between the electrical connector 34 and the respective electrodes 32. In the illustrated embodiment, the electrical connector 34 takes the form of a connector header that is affixed to the top of the plug body 24 using suitable means, such as welding. Alternatively, the electrical connector 34 may be formed as a portion of the plug body 24. The electrical connector 34 includes electrical terminals (not shown) that are external accessible when the hybrid plug/electrode array 12 is anchored within the burr hole 4. In this manner, the respective electrical lead 18 may be mated with the electrical connector 34, such that electrical contacts (not shown) located on the proximal end of the electrical lead contact the electrical terminals of the electrical connector 34. The electrical lead 18 that is coupled to the hybrid plug/electrode array 12 comprises a plurality of insulated wires (not shown)—one for each electrical contact.

The electrode leads 36 extend through the plug body 24 between the respective electrodes 32 to the electrical connector 34 in contact with the electrical terminals. The electrode leads 36 may be suitably coupled to the electrodes 32 and connector 34, e.g., using soldering. If the plug body 24 is composed of an electrically conductive material, each of the electrode leads 36 may have an electrically insulative coating (not shown) to prevent electrical shorting between the electrode leads 36 and the plug body 24. In the illustrated embodiment, the number of electrode leads 36 equals the number of electrodes 32, such that each electrode lead 36 is connected to a respective one of the electrodes 32. In an alternative embodiment, the number of electrode leads 36 may be less or more than the number of electrodes 32. For example, there may be many electrode leads 36 and a single electrode 32, or there may be many electrodes 32 and a single electrode lead 36.

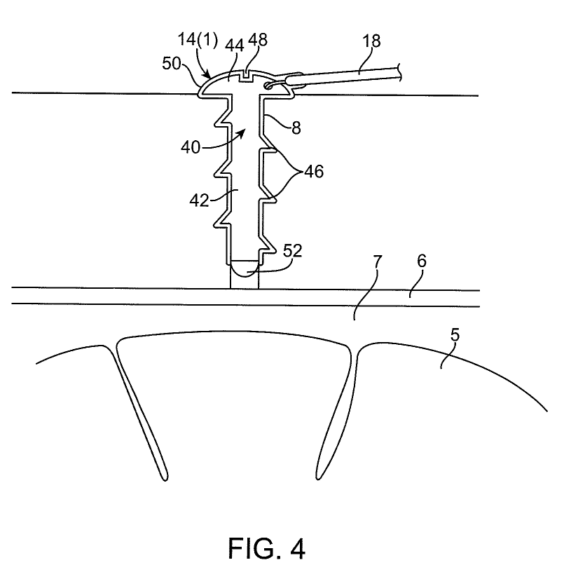

Referring now to FIGS. 4-9, various embodiments of the minimally invasive plug electrodes 14 will now be described. Each of the plug electrodes 14 may be configured for being anchored within a very small burr hole 8 formed within the cranium 3, thereby minimizing the trauma caused to the patient 1. The diameter of the burr hole 8 is preferably less than 10 mm, and more preferably less than 5 mm. Like the hybrid plug/electrode array 12, each plug electrode 14 is sized to be firmly secured within the respective burr hole 8, without extending into the brain of the patient, and in these illustrated cases, without extending within the cranial cavity of the patient.

Referring specifically to FIG. 4, one embodiment of a minimally invasive plug electrode 14(1) will now be described. The plug electrode 14(1) comprises a plug body 40 that includes comprises a shaft 42 configured for being mounted within the burr hole 8 and a head 44 that is externally accessible when the plug body 40 is anchored within the burr hole 8. The plug body 24 comprises a fastener, and in particular, a thread 46 disposed on the outer surface of the shaft 42 for engaging the inner surface of the burr hole 8, and thereby, anchoring the plug electrode 14(1) within the burr hole 8.

The distal end of the shaft 42 is preferably blunt to ensure that the cortical brain tissue 5 is not pierced or otherwise damaged. The plug body 40 includes a tool engagement element 48 for engaging a tool (not shown) that can provide a mechanical advantage for rotation of the plug electrode 14(1). In the illustrated embodiment, the tool engagement element 48 is a slotted recess for receiving a flathead screwdriver. Other types of tool engagement elements, such as a hex recess for receiving a hex wrench, a crossed recess for receiving a Phillips screwdriver, or a bolt head for receiving an open-ended wrench, box-end wrench, or socket wrench can also be used. Thus, it can be appreciated from the foregoing that the plug body 24 takes the form of a screw, which allows the plug electrode 14(1) to be conveniently screwed into the burr hole 4, which may be formed prior to screwing the plug electrode 14(1) therein, or may be formed by screwing the plug electrode 14(1) directed into the cranium 3.

The plug body 40 is composed of a suitable hard and electrically conductive biocompatible material, such as titanium, stainless steel (e.g., MP35N), or alloy. To ensure that the cranium 3 is electrically insulated from the electrically conductive plug body 40 and to minimize noise from electromyograms (EMGs) during recording, the plug electrode 14(1) comprises a durable electrically insulative coating 50 (such as, e.g., epoxy or parylene) disposed on the outer surface of the plug body 24 (including the shaft 42 and head 44). Significantly, the distal end of the shaft 42 is left exposed to form an electrode 52 that faces the cortical brain tissue 5. The plug electrode 14(1) can be considered permanently integrated in that the plug body 40 and electrode 52 are either formed as a unibody design or are otherwise integrated in a manner (e.g., bonding) that would prevent them from being separated from each other without destroying or otherwise damaging the plug electrode 14(1).

As shown, the exposed electrode 52 is recessed within the burr hole 8, and therefore, does not extend into the cranial cavity. Although the exposed electrode 52 is not in direct contact with the cortical brain tissue 5, like the aforementioned electrode array 32 (shown in FIG. 3), it is indirectly electrically coupled to the cortical brain tissue 5 via the dura mater 6 and cerebrospinal fluid 7. Thus, the electrode 52 may potentially convey electrical stimulation energy (originating from the electronics device 16) to the cortical brain tissue 5 or receive electrical signals from the cortical brain tissue 5 for subsequent processing in the electronics device 16. The electrical lead 18 may be connected to the head 42 of the plug body 40 using suitable means, such as soldering, tightening screws, or sutures. Thus, it can be appreciated that the electrical lead 18 is electrically coupled to the exposed electrode 52 via the shaft 42 of the plug body 40.

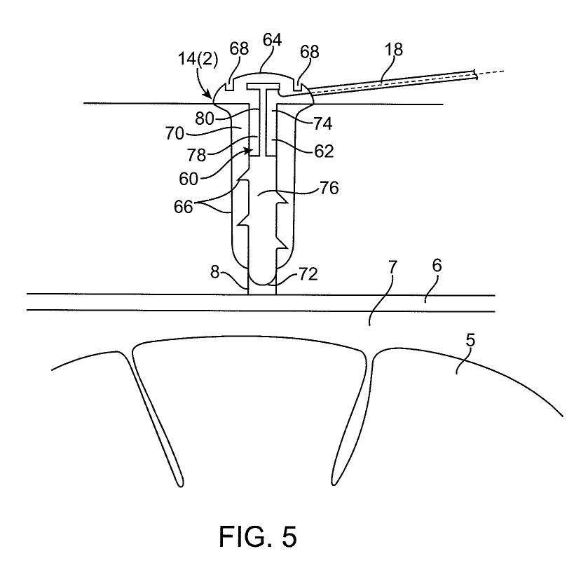

Referring to FIG. 5, another embodiment of a minimally invasive plug electrode 14(2) will now be described. The plug electrode 14(2) is similar to the plug electrode 14(1) illustrated in FIG. 4 in that it is permanently integrated. In particular, the plug electrode 14(2) comprises a plug body 60 that includes a shaft 62 configured for being mounted within the burr hole 8 and a head 64 that is externally accessible when the plug body 40 is anchored within the burr hole 8. The plug body 24 comprises a fastener, and in particular, a thread 66 disposed on the outer surface of the shaft 62 for engaging the inner surface of the burr hole 8, and thereby, anchoring the plug electrode 14(2) within the burr hole 8. In this manner, the plug electrode 14(2) may be conveniently screwed into the burr hole 4 much like the plug electrode 14(1) described above. The distal end of the shaft 62 is preferably blunt to ensure that the cortical brain tissue 5 is not pierced or otherwise damaged. The plug body 40 includes a tool engagement element 68 for engaging a tool (not shown) that can provide a mechanical advantage for rotation of the plug electrode 14(2). In the illustrated embodiment, the tool engagement element 68 a pair of slotted recesses for a special tool. Other types of tool engagement elements, such as those described above, can also be used.

The plug electrode 14(2) mainly differs from the plug electrode 14(1) in that a portion of the plug body 60 is composed of an electrically insulative material. In particular, the plug electrode 14(2) has a top portion 74 (including the head 64 and the proximal end of the shaft 62) that is composed of an electrically insulative material, such as PEEK, and a bottom portion 76 (the distal portion of the shaft 62) that is composed of a suitable hard and electrically conductive biocompatible material, such as titanium, stainless steel (e.g., MP35N), or alloy. The top portion 74 of the plug body 40 comprises a blind lumen 78 that houses an inner electrical conductor 80. The distal end of the blind lumen 78 is open, such that the bottom portion 76 of the plug body 40 (i.e., the electrode) is in electrical communication with the inner conductor 80, and the proximal end of the blind lumen 78 is closed. In the illustrated embodiment, the blind lumen 78, and thus, the inner conductor 80, are T-shaped.

To ensure that the cranium 3 is electrically insulated from the electrically conductive top portion 74 of the plug body 40 and to minimize noise from electromyograms (EMGs) during recording, the plug electrode 14(2) comprises a durable electrically insulative coating 70 (such as, e.g., epoxy or parylene) disposed on outer surface of the shaft 62. Significantly, the distal end of the shaft 62 is left exposed to form an electrode 72 that faces the cortical brain tissue 5. As shown, the exposed electrode 72 is recessed within the burr hole 8, and therefore, does not extend into the cranial cavity. Although the exposed electrode 72 is not in direct contact with the cortical brain tissue 5, like the aforementioned electrode array 32, it is indirectly electrically coupled to the cortical brain tissue 5 via the dura mater 6 and cerebrospinal fluid 7. Thus, the electrode 72 may potentially convey electrical stimulation energy (originating from the electronics device 16) to the cortical brain tissue 5 or receive electrical signals from the cortical brain tissue 5 for subsequent processing in the electronics device 16. The electrical lead 18 may be connected to the inner conductor 80 within the plug body 60, and in particular, the horizontal portion of the inner conductor 80, via a solder or other suitable connection. Thus, it can be appreciated that the electrical lead 18 is electrically coupled to the exposed electrode 72 via the inner conductor 80 and the bottom portion 76 of the plug body 40.

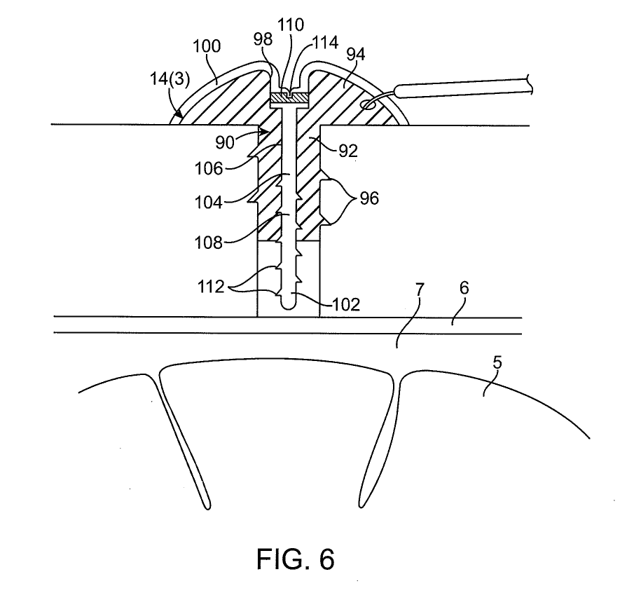

Referring to FIG. 6, yet another embodiment of a minimally invasive plug electrode 14(3) will now be described. The plug electrode 14(3) is similar to the plug electrode 14(1) illustrated in FIG. 4 in that it comprises a plug body 90 that includes a shaft 92 configured for being mounted within the burr hole 8 and a head 94 that is externally accessible when the plug body 90 is anchored within the burr hole 8. The plug body 90 comprises a fastener, and in particular, a thread 96 disposed on the outer surface of the shaft 92 for engaging the inner surface of the burr hole 8, and thereby, anchoring the plug electrode 14(3) within the burr hole 8. In this manner, the plug electrode 14(3) may be conveniently screwed into the burr hole 4. The plug body 90 includes a tool engagement element 98 for engaging a tool (not shown) that can provide a mechanical advantage for rotation of the plug electrode 14(3). In the illustrated embodiment, the tool engagement element is a slotted recess for receiving a flathead screwdriver, although other types of tool engagement elements, such as those described above, can also be used.

The plug electrode 14(3) mainly differs from the plug electrode 14(1) in that it is not permanently integrated. In particular, the plug electrode 14(3) includes an inner electrical conductor 104 concentrically and removably disposed within the plug body 90. In particular, the plug body 90 comprises a lumen 106 extending vertically up the shaft 92 and then out the top of the head 94. The inner conductor 104 takes the form of a screw that includes a shaft 108, which is received within the lumen 106 of the plug body 90, and a head 110 received within the tool engagement element 98 of the plug body 90. The exterior surface of the shaft 104 of the inner conductor 104 and the inner surface of the lumen 106 include threads 112, such that the inner conductor 104 can be screwed into the plug body 40 until the distal end of the shaft 108 of the inner conductor 104, which forms an electrode 102, distally protrudes from the distal end of the plug body 40. The distal ends of the shaft 92 of the plug body 90 and shaft 108 of the inner conductor 104 are preferably blunt to ensure that the cortical brain tissue 5 is not pierced or otherwise damaged. The inner conductor 104 includes a tool engagement element 114 for engaging a tool (not shown) that can provide a mechanical advantage for rotation of the inner conductor 104 within plug body 90. In the illustrated embodiment, the tool engagement element 114 is a slotted recess for receiving a flathead screwdriver, although other types of tool engagement elements, such as those described above, can also be used.

The plug body 90 is composed of a suitable hard and electrically conductive biocompatible material, such as titanium, stainless steel (e.g., MP35N), or alloy. To ensure that the cranium 3 is electrically insulated from the electrically conductive plug body 90 and to minimize noise from electromyograms (EMGs) during recording, the plug electrode 14(3) comprises a durable electrically insulative coating 100 (such as, e.g., epoxy or parylene) disposed on the outer surface of the plug body 90. While only the head 94 of the plug body 90 is shown with the insulative coating 100, the shaft 92 of the plug body 90 may have the insulative coating 100 as well. Alternatively, the plug body 90 may be composed of an electrically insulative material, in which case, the electrically insulative coating 100 may not be needed.

As shown, the electrode 102 is recessed within the burr hole 8, and therefore, does not extend into the cranial cavity. Although the electrode 102 is not in direct contact with the cortical brain tissue 5, like the aforementioned electrode array 32, it is indirectly electrically coupled to the cortical brain tissue 5 via the dura mater 6 and cerebrospinal fluid 7. Thus, the electrode 102 may potentially convey electrical stimulation energy (originating from the electronics device 16) to the cortical brain tissue 5 or receive electrical signals from the cortical brain tissue 5 for subsequent processing in the electronics device 16.

In the case where the plug body 90 is composed of an electrically conductive material, the electrical lead 18 may be connected to the head 94 of the plug body 90 using suitable means, such as soldering, tightening screws, or suturing. In the case where the plug body 90 is composed of an electrically insulative material, the distal end of the electrical lead 18 may be inserted through a lumen (not shown) within the head 94 of the plug body 90 and connected to the inner conductor 104 using suitable means, such as soldering. Thus, it can be appreciated that the electrical lead 18, when connected to the inner conductor 104 (either directly or indirectly through the plug body 90), will be electrically coupled to the exposed electrode 102.

It can be appreciated that the plug electrode 14(3) is particularly advantageous in that the position of the electrode 102 within the burr hole 8 may be adjusted simply by rotating the inner conductor 104 (using the tool). Thus, the electrode 102 can be properly positioned regardless of the thickness of the cranium 3.

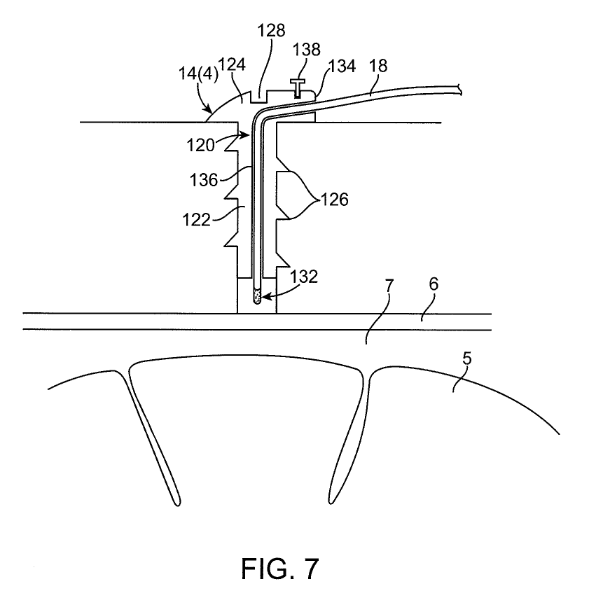

Referring to FIG. 7, yet another embodiment of a minimally invasive plug electrode 14(4) will now be described. The plug electrode 14(4) is similar to the plug electrode 14(3) illustrated in FIG. 6 in that it comprises a plug body 120 that includes a shaft 122 configured for being mounted within the burr hole 8 and a head 124 that is externally accessible when the plug body 120 is anchored within the burr hole 8. The plug body 120 comprises a fastener, and in particular, a thread 126 disposed on the outer surface of the shaft 122 for engaging the inner surface of the burr hole 8, and thereby, anchoring the plug electrode 14(4) within the burr hole 8. In this manner, the plug electrode 14(4) may be conveniently screwed into the burr hole 4. The plug body 120 includes a tool engagement element 128 for engaging a tool (not shown) that can provide a mechanical advantage for rotation of the plug electrode 14(4). In the illustrated embodiment, the tool engagement element is a slotted recess for receiving a flathead screwdriver, although other types of tool engagement elements, such as those described above, can also be used.

The plug electrode 14(4) mainly differs from the plug electrode 14(3) in that, instead of having a screw-like inner electrical conductor, the electrical lead 18, itself, is removably disposed within the plug body 120. In particular, the plug body 120 comprises a lumen 136 extending vertically up the shaft 62 and then horizontally out of the head 124. The electrical lead 18, which carries an electrode 132 at its distal end, is configured to firmly slide within the lumen 136, such that the electrical lead 18 can be threaded into an opening 134 at the head 124 until the electrode 132 distally protrudes from the distal end of the plug body 120. To this end, the diameter of the lumen 136 is substantially equal to the outer diameter of the electrical lead 18. The distal end of the shaft 122 of the plug body 120 and the distal end of the electrical lead 18 are preferably blunt to ensure that the cortical brain tissue 5 is not pierced or otherwise damaged. The plug electrode 14(4) further comprises a tightening screw 138 that can be screwed into the top of the head 124 to firmly secure the electrical lead 18 once it is confirmed that the electrode 132 is in its proper place.

To ensure that the cranium 3 is electrically insulated from the plug electrode 14(4) and to minimize noise from electromyograms (EMGs) during recording, the plug body 120 may be composed of an electrically insulative material, and the electrical lead 18, with the exception of its distal end, can be coated within an electrically insulative material. As shown, the exposed electrode 132 is recessed within the burr hole 8, and therefore, does not extend into the cranial cavity. Although the exposed electrode 132 is not in direct contact with the cortical brain tissue 5, like the aforementioned electrode array 32, it is indirectly electrically coupled to the cortical brain tissue 5 via the dura mater 6 and cerebrospinal fluid 7. Thus, the electrode 132 may potentially convey electrical stimulation energy (originating from the electronics device 16) to the cortical brain tissue 5 or receive electrical signals from the cortical brain tissue 5 for subsequent processing in the electronics device 16. It should be appreciated that the plug electrode 14(4) is particularly advantageous in that the position of the electrode 132 within the burr hole 8 may be adjusted simply by sliding the electrical lead 18 within the lumen 136 of the plug body 120 when the tightening screw 138 is loosened. Thus, the electrode 132 can be properly positioned regardless of the thickness of the cranium 3.

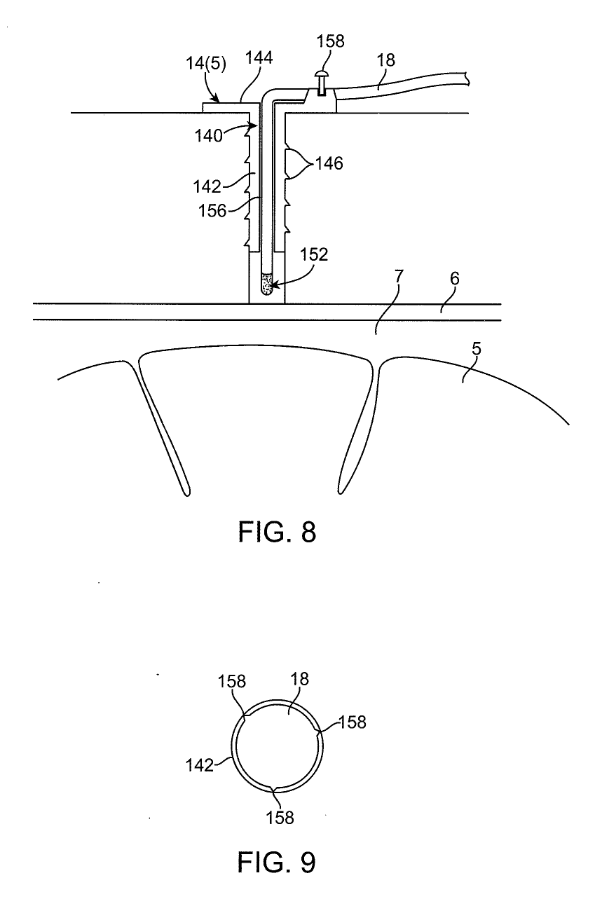

Referring to FIG. 8, yet another embodiment of a minimally invasive plug electrode 14(5) will now be described. The plug electrode 14(5) is similar to the plug electrode 14(4) illustrated in FIG. 7 in that it comprises a plug body 140 capable of sliding receiving the electrical lead 18. Thus, the plug body 140 comprises a shaft 142 configured for being mounted within the burr hole 8 and a head 144 that is externally accessible when the plug body 140 is anchored within the burr hole 8. The plug body 140 comprises a lumen 156 extending vertically up the shaft 142. The electrical lead 18, which carries an electrode 152 at its distal end, is configured to firmly slide within the lumen 156, such that the electrical lead 18 can be threaded into an opening 154 at the head 144 until the electrode 152 distally protrudes from the distal end of the plug body 140. To this end, the diameter of the lumen 156 is substantially equal to the outer diameter of the electrical lead 18. The distal ends of the plug base shaft 62 and electrical lead 18 are preferably blunt to ensure that the cortical brain tissue 5 is not pierced or otherwise damaged. The plug electrode 14(5) also comprises a tightening screw 158 that can be screwed into the top of the head 144 to firmly secure the electrical lead 18 once it is confirmed that the electrode 152 is in its proper place.

To ensure that the cranium 3 is electrically insulated from the electrically conductive plug body 140 and to minimize noise from electromyograms (EMGs) during recording, the plug body 140 may be composed of an electrically insulative material, and the electrical lead 18, with the exception of its distal end, can be coated within an electrically insulative material. As shown, the exposed electrode 152 is recessed within the burr hole 8, and therefore, does not extend into the cranial cavity. Although the exposed electrode 152 is not in direct contact with the cortical brain tissue 5, like the aforementioned electrode array 32, it is indirectly electrically coupled to the cortical brain tissue 5 via the dura mater 6 and cerebrospinal fluid 7. Thus, the electrode 152 may potentially convey electrical stimulation energy (originating from the electronics device 16) to the cortical brain tissue 5 or receive electrical signals from the cortical brain tissue 5 for subsequent processing in the electronics device 16. It should be appreciated that the plug electrode 14(5) is particularly advantageous in that the position of the electrode 52 within the burr hole 8 may be adjusted simply be sliding the electrical lead 18 within the lumen 156 of the plug body 140 when the tightening screw 158 is loosened. Thus, the electrode 152 can be properly positioned regardless of the thickness of the cranium 5.

The plug electrode 14(5) mainly differs from the plug electrode 14(4) in that it uses a different fastening means for anchoring the plug body 140 within the burr hole 8. In particular, the plug electrode 14(5) comprises a series of annular ribs 146 formed on the external surface of the plug body 140, such that when the plug electrode 14(5) is inserted within the burr hole 8, the annular ribs 146 grasp the burr hole 8, thereby firmly securing the plug electrode 14(5) within the burr hole 8. Unlike the plug electrode 14(4), no tool is needed to anchor the plug electrode 14(5) into the burr hole 8. The electrical lead 18 also comprises a plurality of vertical ribs 158 (as best illustrated in FIG. 9) that facilitate engagement within the lumen 156 of the plug body 140.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A method of performing a medical procedure on a patient, comprising:
forming a burr hole through the cranium of the patient;
mounting a permanently integrated plug electrode within the burr hole; and
electrically coupling the plug electrode to an electronics device.

2. The method of claim 1, wherein the burr hole has a diameter of 10 mm or less.

3. The method of claim 1, wherein the burr hole has a diameter of 5 mm or less.

4. The method of claim 1, wherein mounting the plug electrode within the burr hole comprises screwing the plug electrode within the burr hole.

5. The method of claim 1, wherein the plug electrode has at least one electrode formed on an external surface of the plug electrode.

6. The method of claim 5, wherein the at least one electrode contact comprises a plurality of electrodes.

7. The method of claim 1, wherein the plug electrode has at least one electrode disposed within the burr hole when the plug electrode is mounted within the burr hole.

8. The method of claim 1, wherein a proximal portion of the plug electrode is electrically insulated from the patient, and a distal portion of the plug electrode is exposed to form at least one electrode.

9. The method of claim 1, wherein forming the burr hole within the cranium of the patient and mounting the plug electrode within the burr hole comprises screwing the plug electrode directly into the cranium of the patient.

10. The method of claim 1, wherein electrically coupling the plug electrode to the electronics device comprises connecting an electrical lead between the plug electrode and the electronics device.

11. The method of claim 1, further comprising implanting the electronics device within the patient.

12. The method of claim 1, wherein the electronics device is a neurostimulator, the method further comprising:
forming another burr hole through the cranium of the patient;
mounting another plug electrode within the burr hole; and
electrically coupling the other plug electrode to an electrical signal recorder.

13. A method of performing a medical procedure on a patient, comprising:
forming a burr hole through the cranium of the patient;
mounting an electrode within the burr hole, such that the electrode does not extend into a brain of the patient; and
electrically coupling the electrode to an electronics device.

14. The method of claim 13, wherein the burr hole has a diameter of 10 mm or less.

15. The method of claim 13, wherein the burr hole has a diameter of 5 mm or less.

16. The method of claim 13, further comprising mounting a plug within the burr hole to secure the electrode within the burr hole.

17. The method of claim 16, wherein the plug and electrode form a permanently integrated plug electrode that is mounted within the burr hole to secure the electrode within the burr hole.

18. The method of claim 16, wherein the electrode is carried by a lead, and the lead is affixed within the plug to secure the electrode within the burr hole.

19. The method of claim 13, wherein the electrode is mounted within the burr hole, such that the electrode does not extend into a cranial cavity of the patient.

20. The method of claim 13, further comprising implanting the electronics device within the patient.

21. The method of claim 13, wherein the electronics device is a neurostimulator, the method further comprising:
forming another burr hole through the cranium of the patient;
mounting another electrode within the other burr hole, such that the other electrode does not extend within a cranial cavity of the patient; and
electrically coupling the other electrode to an electrical signal recorder.

22. A hybrid plug/electrode, comprising:
a plug body configured for being anchored within a burr hole formed within a cranium of a patient, the plug body having a distal-facing surface;
at least one electrode disposed on the distal-facing surface of the plug body; and
at least one electrode lead affixed within the plug body in electrical communication with the at least one electrode.

23. The hybrid plug/electrode of claim 22, wherein the plug body comprises a cylindrical outer wall configured for engaging an inner surface of the burr hole.

24. The hybrid plug/electrode of claim 23, wherein the plug body comprises at least one fastening mechanism disposed on the cylindrical outer wall for anchoring the plug body to the inner surface of the burr hole.

25. The hybrid plug/electrode of claim 24, wherein the at least one fastening mechanism is a thread.

26. The hybrid plug/electrode of claim 22, wherein the at least one electrode comprises a plurality of electrodes.

27. The hybrid plug/electrode of claim 26, wherein the at least one electrode lead comprises a plurality of electrode leads.

28. The hybrid plug/electrode of claim 22, further comprising a connector affixed to the plug body in electrical communication with the at least one electrode lead, the connector configured for being externally accessible when the plug body is anchored within the burr hole.

29. The hybrid plug/electrode of claim 28, wherein the connector is configured for receiving a lead extension.

30. A medical system, comprising:
the hybrid plug/electrode of claim 22; and
an electronics device coupled to the one or more electrode leads.

31. A method of performing a medical procedure on a patient, comprising:
forming the burr hole through the cranium of the patient;
anchoring the hybrid plug/electrode of claim 22 within the burr hole; and
electrically coupling the hybrid plug/electrode to an electronics device.

32. The method of claim 31, wherein the at least one electrode does not extend within a cranial cavity of the patient when the hybrid plug/electrode is anchored within the burr hole.

33. A method of performing a medical procedure on a patient, comprising conveying electrical energy from the at least one electrode of the hybrid plug/electrode of claim 22 to stimulate cortical brain tissue of the patient.

34. A method of performing a medical procedure on a patient, comprising recording electrical signals from the cortical brain tissue using the at least one electrode of the hybrid plug/electrode of claim 22.
